(19) United States
(12) Patent Application Publication
(10) Pub. No.: US 2020/0390930 A1
(43) Pub. Date: Dec. 17, 2020

(54) SYSTEMS AND METHODS FOR TREATING A WOUND WITH WOUND PACKING

(71) Applicants: Brock Liden, Baltimore, OH (US); Spencer Brown, Leesburg, IN (US)

(72) Inventors: Brock Liden, Baltimore, OH (US); Spencer Brown, Leesburg, IN (US)

(21) Appl. No.: 16/798,180

(22) Filed: Feb. 21, 2020

Related U.S. Application Data
(63) Continuation of application No. 15/620,531, filed on Jun. 12, 2017, now abandoned.
(60) Provisional application No. 62/348,698, filed on Jun. 10, 2016.

(51) Int. Cl. A61L 15/40; A61L 26/00; A61F 13/00; A61L 15/32

(52) U.S. Cl. CPC A61L 15/40; A61L 26/0033; A61L 15/325; A61F 13/00063; A61F 13/00029; A61L 26/0057

(57) ABSTRACT

Methods for treating a wound with a wound packing are discussed. While the wound packing can include any suitable component, in some cases, it includes a collection of multi-potent cells (e.g., cells from bone marrow, amniotic membrane tissue, amniotic fluid, stem cells, etc.), plasma (e.g., concentrated and/or platelet rich plasma), and collagen (e.g., native and/or organized reconstituted collagen). In some cases, the wound packing is gelled, coagulated, or otherwise hardened through the use of thrombin, calcium chloride, and/or another suitable additive. In some cases, the wound packing is shaped to substantially correspond to the wound's shape. While the wound packing can be used in any suitable manner, in some instances, it is applied to the wound, skin fragments are applied to the packing, the packing is secured to the wound, and/or the packing is covered with a protective barrier. Other implementations are also described.

### RELATED APPLICATIONS

This application is a continuation of U.S. patent application Ser. No. 15/620,531 (Attorney Docket No. 28973.4), filed Jun. 12, 2017, and entitled “SYSTEMS AND METHODS FOR TREATING A WOUND WITH WOUND PACKING,” which claims priority to U.S. Provisional Patent Application Ser. No. 62/348,698 (Attorney Docket No. 22996.3), filed Jun. 10, 2016, and entitled “SYSTEMS AND METHODS FOR TREATING A WOUND WITH A WOUND PACKING;” each of which is incorporated herein by reference in its entirety.

### BACKGROUND OF THE INVENTION

### 1. Field of the Invention

The present invention relates to systems and methods for treating a wound. In particular, some implementations of the present invention relate to methods for forming a wound packing comprising multi-potent cells (e.g., bone marrow cells), plasma, collagen, and/or a thickening agent. In some further implementations, once the wound packing is applied to a wound, skin seeds are optionally applied to a surface of the packing and a protective barrier is applied over the packing.

### 2. Background and Related Art

Many people across the world suffer from skin ulcerations and injuries. For instance, many people who suffer from diabetes, who are bedridden, or who are confined to a wheelchair can also suffer from poor blood flow in some portions of their bodies, such as their appendages. As a result, such people can be at a relatively high risk of developing sores, ulcers, and other wounds on their body. While these wounds can form on virtually any portion of a person's body, some people develop particularly chronic sores, ulcers, and/or other wounds on their legs and feet—especially on the soles of their feet.

In any case, chronic sores, ulcers, and other wounds can dramatically affect an individual's life, limiting the individual's ability to move (e.g., walk), work, and play. Moreover, such wounds can cause pain and take an emotional toll on the individual, costing the individual and/or others relatively large amounts of money, energy, and time spent in, and for, recovery. Additionally, in some extreme cases, such sores, ulcers, and other wounds can lead to amputation, which can dramatically complicate life, be physically and emotionally draining, increase healthcare costs, and otherwise be extremely undesirable.

While a variety of treatments, ointments, bandages, medical procedures, and other systems and methods have been used to treat chronic wounds, such systems and methods are not necessary without their shortcomings. Indeed, some methods and treatments for caring for persistent wounds may be relatively ineffective at treating chronic wounds. As a result, some systems and methods for treating such wounds may never actually result in the wound closing and healing. In some other cases, while some conventional systems and methods may help a chronic wound to heal, some such systems and methods can be relatively slow—drawing out treatment and recovery time over a relatively long period of time.

Thus, while techniques currently exist that are used to treat chronic ulcerations, sores, and other wounds, challenges still exist, including those mentioned above. Accordingly, it would be an improvement in the art to augment or even replace current techniques with other techniques.

### SUMMARY OF THE INVENTION

The present invention relates to systems and methods for treating a wound. In particular, some implementations of the present invention relate to methods for forming a wound packing comprising multi-potent cells (e.g., bone marrow cells), plasma, collagen, and/or a thickening agent. In some further implementations, once the wound packing is applied to a wound, skin seeds are optionally applied to a surface of the packing and a protective barrier is applied over the packing.

While the described methods can comprise any suitable step or characteristic, in some implementations, the described methods include combining multi-potent cells (e.g., cells from bone marrow, amniotic tissue, amniotic fluid, placental tissue, fat tissue, umbilical cord tissue, stem cells, and/or any other suitable source) with plasma (e.g., platelet rich plasma, concentrated plasma, super concentrated plasma, plasma, and/or any other suitable type of plasma), and collagen (e.g., native collagen, organized reconstituted collagen, collagen, and/or any other suitable type of collagen). In some implementations, the various components are formed into a shape that substantially corresponds to the shape of a wound. Additionally, in some implementations, thrombin, calcium chloride, one or more serine proteases, and/or any other suitable thickening agent or agents are combined with the multi-potent cells, plasma, and/or collagen to coagulate, gel, and/or otherwise harden the components to form a wound packing.

In some implementations, the wound packing is inserted into or otherwise applied to a wound (e.g., a wound that has been cleaned, debrided, disinfected, had necrotic tissue removed, that is bleeding, and/or that is otherwise ready to receive the packing). Moreover, in some such implementations, “skin seeds” or skin graft fragments (e.g., micronized and/or otherwise fragmented epidermal and/or dermal tissue) are applied to the wound packing (e.g., an outer surface of the wound packing). Additionally, in some cases, the wound, wound packing, and skin cells are covered with a protective barrier. In this regard, some non-limiting examples of such barriers include a single layer protective membrane, such as a layer of a-cellular dermis (e.g., human a-cellular dermis, animal dermis, etc.), a layer of silicon (e.g., one or more layers of a surgical silicone), and/or any other suitable single layer membrane; a bilayer protective membrane, such as a piece of an a-cellular dermis with a basement membrane, a piece of an a-cellular dermis comprising a layer of surgical silicone, and/or any other suitable bi-layer membrane; and/or any other suitable membrane comprising two or more layers.

In some implementations, once the wound packing has been placed in and/or on a wound, the wound is dressed further. While the wound can be dressed in any suitable manner, in some cases, the wound is dressed with: gauze, a bandage, a wrap, a total contact cast, a non-weight-bearing cast or dressing, a wound veil, a contact layer flex bandage, a compressive dressing, KERLIX™ wrapping, an absorbent abdominal pad, calcium alginate, a hydra fiber, a fenestrated gauze drain sponge, a TRIACT™ treatment (e.g., produced by Hollister of Libertyville, Ill., USA), an ointment, a petroleum-based jelly, and/or with any other suitable type of wound dressing.

As mentioned, the described systems and methods can be modified in any suitable manner. Indeed, in some implementations, the wound packing lacks multi-potent cells, while still comprising plasma and collagen. In some other implementations, the wound packing lacks collagen. In still other non-limiting implementations, the wound packing is used without the application of skin seeds to an external surface of the packing. In still other implementations, one or more components of the wound packing (e.g., the multi-potent cells, the plasma, skin seeds, etc.) are autologous to the person to which the wound packing is to be applied. In yet other implementations, however, one or more components of the wound packing are autologous, allogeneic, xenogeneic, and/or syngeneic to the person to which the wound packing is to be applied.

While the described systems and methods may be particularly useful in the area of treating one or more sores, ulcerations, and/or other wounds on the sole (and other portions) of an individual's foot, those skilled in the art can appreciate that the described systems and methods can be used in a variety of different applications and in a variety of different areas of manufacture to treat a wide range of injuries, in any suitable location, including, without limitation, injuries to an individual's arm, hand, finger, toe, leg, thigh, calf, buttocks, back, torso, abdomen, neck, head, face, etc.). Indeed, in some implementations, the described systems and methods are used to treat surgical wounds, bed sores, pressure ulcers, wound dehiscence, graft donor sites, lesions, sores, vascular ulcers, diabetic ulcers (including, without limitation, diabetic foot ulcers), trauma wounds, tears, tunneling wounds, abrasions, sores, lacerations, and/or other wounds.

These and other features and advantages of the present invention will be set forth or will become more fully apparent in the description that follows. Furthermore, the features and advantages of the invention may be learned by the practice of the invention or will be obvious from the description, as set forth hereinafter.

### BRIEF DESCRIPTION OF THE DRAWINGS

In order that the manner in the above-recited and other features and advantages of the present invention are obtained, a more particular description of the invention will be rendered by reference to specific embodiments thereof, which are illustrated in the appended drawings. Understanding that the drawings depict only typical embodiments of the present invention and are not, therefore, to be considered as limiting the scope of the invention, the present invention will be described and explained with additional specificity and detail through the use of the accompanying drawings in which:

FIG. 1 illustrates a flowchart depicting a method for forming a wound packing in accordance with a representative embodiment;

FIGS. 2A-2D each illustrate a cross-sectional view of a different representative embodiment of the wound packing;

FIG. 3 illustrates a flowchart depicting a method for treating a wound with the wound packing in accordance with a representative embodiment; and

FIG. 4 illustrates a cross-sectional view of the wound packing applied to a wound in accordance with a representative embodiment.

### DETAILED DESCRIPTION OF THE INVENTION

The present invention relates to systems and methods for treating a wound. In particular, some implementations of the present invention relate to methods for forming a wound packing comprising multi-potent cells (e.g., bone marrow cells), plasma, collagen, and/or a thickening agent. In some further implementations, once the wound packing is applied to a wound, skin seeds are optionally applied to a surface of the packing and a protective barrier is applied over the packing.

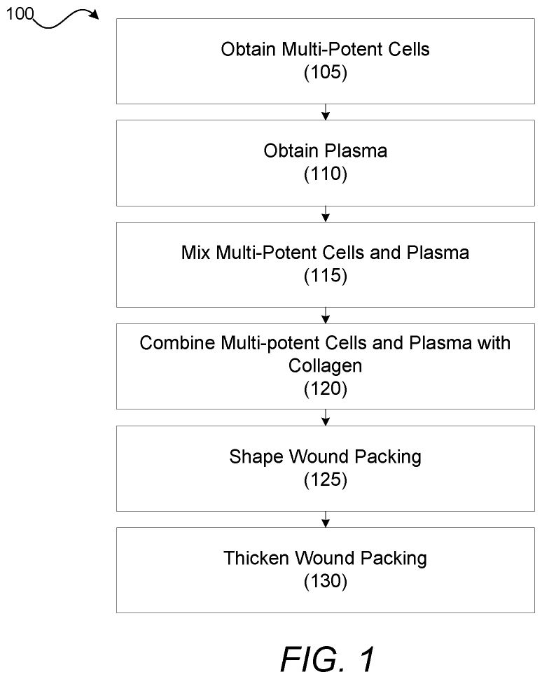

In accordance with some embodiments, the described wound packing and its associated methods are configured to help wounds (including chronic wounds) close up and otherwise heal. While the wound packing can be formed in any suitable manner, FIG. 1 illustrates a representative embodiment of a method 100 for forming the described wound packing. While such method 100 (and all other methods describe herein) can be modified in any suitable manner (e.g., with any suitable portion of the method being omitted, added to, reordered, performed simultaneously with another portion of the method, performed independently, substituted with another technique, and/or otherwise being modified in any suitable manner), step 105 in FIG. 1 shows that, in some embodiments, the method 100 includes obtaining multi-potent cells.

Where the wound packing comprises multi-potent cells, the multi-potent cells can comprise any suitable cells that are capable of differentiating and helping a wound to which they are applied to heal. In this regard, the term multi-potent cells may be used herein to refer to cells that are totipotent, pluripotent, and/or multipotent. Some non-limiting examples of such cells include bone marrow cells, mesenchymal cells, stem cells, neural stem cells, cells from amniotic fluid, placental cells, amniotic membrane cells, and/or any other suitable unspecialized (e.g., multi-potent and/or undifferentiated) cells that can differentiate into specialized cells with specific functions that are useful in the wound packing.

The multi-potent cells can be obtained from any suitable organism, including, without limitation, from the patient to whom the wound packing is to be applied (e.g., autologous cells), from one or more third-party individuals (e.g., allogenic cells), from an identical twin (e.g., syngeneic cells), from an animal of a different species (e.g., xenogeneic cells), etc. In some embodiments, however, the multi-potent cells comprise autologous cells taken from the patient. As used herein, the terms patient and individual (and variations thereof) may be used to refer to any person or other subject to whom the described wound packing can be applied.

The multi-potent cells can be obtained from any suitable source, including, without limitation, from bone marrow (including, without limitation, mesenchymal cells), placental tissue, amniotic fluid, amniotic membrane tissue, umbilical cord tissue, brain tissues, blood vessels, skeletal muscle, skin, teeth, heart tissue, liver tissue, gut tissue, etc. Moreover, the multi-potent cells can be obtained from any suitable location. Indeed, in some embodiments in which the multi-potent cells comprise bone marrow cells, the bone marrow cells are collected from one or more iliac crests, calcanei, sternums, lumbar vertebrae, femurs, tibias, and/or other suitable locations, from one or more individuals.

The multi-potent cells can be obtained in any suitable manner, including, without limitation, via aspiration (e.g., with a bone marrow aspirate needle or otherwise), biopsy (e.g., trephine biopsy or otherwise), excision, and/or in any other suitable manner. Indeed, in some embodiments, the multi-potent cells comprise bone marrow aspirate taken from the iliac crest, calcaneus, sternum, and/or tibia of the patient to whom the wound packing is to be applied. In such embodiments, the cells can be aspirated in any suitable manner, including, without limitation, via a bone marrow aspirate needle and syringe coated with a blood thinner (e.g., heparin, anti-coagulate citrate dextrose, and/or any other suitable thinner).

In order to form the wound packing, any suitable amount of material comprising multi-potent cells can be extracted from an organism (e.g., the patient). Indeed, in some embodiments in which the multi-potent cells comprise bone marrow cells, between about 1 and about 300 mls of bone marrow aspirate are initially collected. In other embodiments, any suitable amount of multi-potent cells (e.g., bone marrow aspirate) that falls in a sub-range (e.g., between about 40 mls and about 200 mls) of the aforementioned range can be extracted for use in formation in

In some embodiments, the multi-potent cells are optionally concentrated for inclusion in the wound packing. In this regard, the multi-potent cells (e.g., bone marrow cells) can be concentrated in any suitable manner, including, without limitation, via a conventional or novel cell concentration kit (e.g., a BIOCUE™ kit, produced by Biomet Biologics, LLC of Warsaw, Ind., USA) centrifugation, filtration, separation, and/or any other conventional or novel technique that is suitable for concentrating cells for use in the wound packing.

In some embodiments, the multi-potent cells are concentrated through centrifugation. In such embodiments, the centrifugation can be performed in any suitable manner, including, without limitation, by spinning the collected multi-potent cells at between about 60 and about 20,000 rpm for between about 10 seconds and about 120 minutes. Moreover, the multi-potent cells can be centrifuged at any suitable sub-range or sub-ranges of the aforementioned ranges. Indeed, in some embodiments, the multi-potent cells are centrifuged at between about 1,000 and about 6,000 rpm for between about 4 minutes and about 30 minutes. In still other embodiments, the multi-potent cells are centrifuged at between about 2,800 and about 4,000 rpm for between about 10 and about 20 minutes.

While the multi-potent cells can be brought to any suitable concentration, in some embodiments, the concentrated multi-potent cells are between about 2 and about 30 times more concentrated than are such cells when they are initially obtained (including with any blood thinner used in the collection process). In some embodiments, the concentrated multi-potent cells can be concentrated to any suitable sub-range of the aforementioned concentration (including, without limitation, between about 5 and about 15 times as concentrated as the cells when they are originally collected). In one example, when 60 or 120 mls of bone marrow aspirate are drawn from one or more individuals, the bone marrow cells are concentrated down to a volume of about 6 (±2) mls and about 12 (±4) mls, respectively. Said differently, while the multi-potent cells (e.g., bone marrow cells) can be concentrated down to any suitable concentration, in some embodiments, the cells are concentrated to between about 1 cell/ml and about 100 billion cells/ml (or any suitable sub-range thereof). Indeed, while in some embodiments, the multi-potent cells are concentrated to between about 100 cells/ml and about 5 million cells/ml, in some other embodiments, the multi-potent cells are concentrated to between about 1,000 cells/ml and about 1 million cells/ml. In still other embodiments, the multi-potent cells are concentrated at any concentration falling within any of the foregoing ranges.

In some embodiments, once the multi-potent cells (e.g., bone marrow cells) have been concentrated (e.g., via centrifugation), the cells are separated from other materials (e.g., supernatant). In this regard, the multi-potent cells and other material (e.g., supernatant) can be separated from each other in any suitable manner, including, but not limited to, decantation, vacuuming, and/or any other suitable method.

Continuing with the method 100, step 110 shows that, in some embodiments, the method includes obtaining plasma. In this regard, the plasma can be collected form any suitable source, including, but not limited to, one or more autologous, allogenic, xenogeneic, and/or syngeneic cell sources). In some embodiments, however, the plasma is collected from the patient to whom the wound packing is to be applied.

The plasma can be collected in any suitable manner, including, without limitation, by having blood drawn (e.g., with a syringe or other vessel comprising a blood thinner, such as heparin, anti-coagulate citrate dextrose (“ACDA”), and/or any other suitable material) from the patient and/or one or more other individuals, and then extracting plasma (e.g., platelet rich plasma (“PRP”), concentrated plasma, plasma, platelet concentrate, super concentrated plasma, growth factors, etc.) from the blood. In this regard, the plasma (e.g., PRP, concentrated plasma, etc.) can be extracted from collected blood in any suitable manner. Some examples of suitable methods for extracting plasma include, but are not limited to, the use of conventional plasma extraction kits (e.g., a plasma and serum preparation kit provided by Life Technologies of Grand Island N.Y., USA), a plasma concentration kit (e.g., a PLASMAX® and/or a PLASMAX® Plus plasma concentration system, produced by Biomet Biologics, LLC of Warsaw, Ind., USA); a Harvest PRP kit, produced by Harvest Technologies, Corp. of Plymouth Mass., USA; etc.), centrifugation of whole blood and collecting the plasma supernatant, centrifugation of plasma supernatant to obtain platelet concentrate, and/or any other technique. In some embodiments, however, a plasma concentration kit is used to obtain PRP and plasma that is relatively rich with growth factors (e.g., patient derived growth factors).

In order to prepare the wound packing, plasma (e.g., PRP) can be collected from any suitable amount of blood. In some embodiments, however, the plasma is extracted and/or concentrated from between about 10 and about 500 mls or more of blood. In other embodiments, the plasma is concentrated from any suitable sub-range of the about 10 and about 500 mls of blood (e.g., between about 40 and about 80 mls). Indeed, in some embodiments, the plasma is extracted from between about 50 and about 56 mls of blood.

The plasma can be concentrated to any suitable concentration, including, without limitation, to be between about 2 and about 30 times more concentrated than it was in the blood from which the plasma is obtained (including with any blood thinner and/or other materials used in the collection process). Moreover, in some embodiments, the plasma is concentrated to any suitable sub-range of the aforementioned concentration (including, without limitation, between about 5 and about 15 times as concentrated as the cells when they are originally collected). In one example, when about 55 mls of blood are combined with about 5 mls of blood thinner, about 6 mls (±2 mls) of PRP is produced). Said differently, while the plasma can be concentrated down to any suitable concentration, in some embodiments, the plasma is concentrated to between about 1_{E}⁻¹⁰ g/ml and about 3 g/ml (or any suitable sub-range thereof). Indeed, while in some embodiments, the plasma is concentrated to between about 0.001 g/ml and about 2 g/ml, in some other embodiments, the plasma is concentrated to between about 0.1 g/ml and about 1 g/ml (or any subrange of the foregoing ranges.

The plasma (e.g., PRP) can be collected and/or concentrated at any suitable time before being applied to the wound in the wound packing. In some embodiments, however, the blood is collected and/or concentrated within about 4 hours of the time in which the packing is applied to the wound (or any suitable subrange of such time period). In other embodiments, the plasma is concentrated within about an hour (e.g., between about 5 minutes and about 40 minutes) of the packing's application.

Continuing with the method, step 115 shows that the multi-potent cells (e.g., bone marrow concentrate) and plasma (e.g., PRP, plasma concentrate, etc.) are combined. In this regard, the multi-potent cells (concentrated bone marrow aspirate) and plasma (e.g., PRP) can be mixed together at any suitable ratio. Indeed in some embodiments, the concentrated multi-potent cells (e.g., bone marrow aspirate cells) are combined with the plasma (e.g., PRP and/or plasma concentrate) at a ratio between about 100:1 and about 1:100, by volume. In other embodiments, the multi-potent cells and plasma are combined in any suitable sub-range of the aforementioned range (e.g., at a ratio of between about 1:1 and about 6:1). Indeed, in some embodiments, the bone marrow concentrate and PRP and/or plasma concentrate are mixed at a ratio between about 1.5:1 and about 4:1. In an example of such, about 5 cc of concentrated bone marrow aspirate are combined with about 3 cc of concentrated plasma.

Continuing with FIG. 1, step 120 shows that, in some embodiments, the method 100 optionally includes adding the multi-potent cells and plasma to collagen and/or another suitable material. In this regard, the multi-potent cells, plasma, and collagen can be mixed and/or otherwise combined with each other in any suitable manner. In some embodiments, two or more of the components of the packing are mixed via pipetting, a mixer (e.g., a vortex mixer, etc.), a shaker (e.g., an orbital shaker, etc.), an ultrasonic bath or probe, diffusion, adsorption, the application of pressure to the collagen in a bath of one or more of the other described ingredients, and/or in any other suitable manner.

The various components of the wound packing can be combined in any suitable order. Indeed, in some embodiments, the multi-potent cells (e.g., concentrated bone marrow aspirate) and plasma (e.g., PRP) are mixed together before being added to the collagen. In some other embodiments, either the multi-potent cells or the plasma is added to the collagen before the other of the two is. In one such example, the multi-potent cells are placed in a vessel (e.g., a cup, bowl, mold, and/or other container that resembles (slightly or substantially) a shape of the wound), the collagen is added to the cells, and the plasma (e.g., PRP) is then added to the collagen and multi-potent cells. In still other embodiments, the multi-potent cells and plasma are mixed with the collagen simultaneously. In still other embodiments, the collagen is added to the multi-potent cells and/or the plasma.

With respect to the collagen, the collagen can comprise any collagen that is suitable for use in the wound packing. In some embodiments, the collagen comprises a native collagen matrix, organized reconstituted collagen, reconstituted collagen, a collagen structure (e.g., native collagen) without an intact basement membrane, and/or a collagen structure with a fragmented basement membrane from one or more sheep, pigs, horses, humans, and/or other organisms. Indeed, in some embodiments, the collagen comprises one or more layers of a native collagen matrix (e.g., a collagen without a basement membrane or with a fractured basement membrane) taken from a sheep or human.

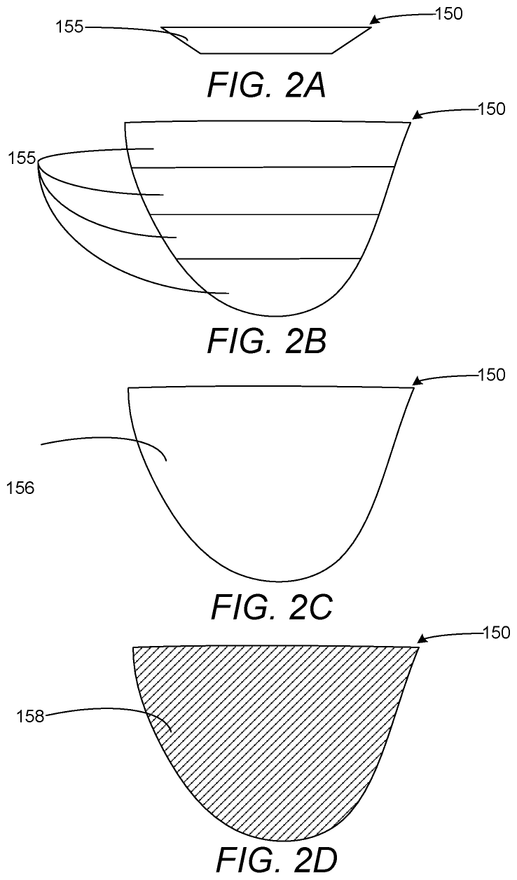

Although, in some embodiments, the collagen comprises reconstituted collagen particulates, in some other embodiments, it comprises 1, 2, 3, 4, 5, 6, 7, 8, 9, or more layers of collagen matrix, depending on the depth of the wound to which the packing is to be applied. By way of non-limiting illustration, FIG. 2A shows an embodiment of the wound packing 150 that is configured for use in a relatively shallow wound and in which the wound packing 150 comprises a single layer of collagen 155. In contrast, FIG. 2B shows an embodiment in which the wound packing 150 comprises 4 layers of collagen 155, for use in a relatively deep wound. Additionally, FIGS. 2C and 2D respectively illustrate some embodiments in which the wound packing 150 comprises reconstituted collagen 156 and a collagen structure 158.

Returning to FIG. 1, step 125 shows that, in some embodiments, the wound packing 150 is shaped such that it is able to fit in a wound, or, in some embodiments, to substantially correspond in shape to an interior shape of the wound. While the packing can be shaped in any suitable manner, in some embodiments, the collagen in the packing is cut, shaved, torn, ripped, trimmed, separated along perforated lines, packed, molded, layered, chamfered, and/or otherwise formed into any desired shape (e.g., a shape that resembles an interior shape of a wound). Similarly, the other ingredients of the wound packing can be shaped in any suitable manner, including, without limitation, by being added a mold, a cup, and/or another container having a shape that resembles that of the wound, by being molded manually, and/or by being added to the collagen, with the collagen being shaped to fit the wound.

Continuing with FIG. 1, step 130 shows that, in some embodiments, the wound packing 150 is gelled, thickened, and/or otherwise hardened. In this regard, the wound packing (e.g., multi-potent cells, plasma, and/or collagen) can be hardened in any suitable manner, including, without limitation, through the application of a thickening agent. Some examples of suitable thickening agents include, but are not limited to, thrombin, recombinant thrombin, calcium chloride, and/or any other suitable ingredient or ingredients that are capable of causing the wound packing to gel and/or otherwise harden.

Where a thickening agent is added to the wound packing 150, any suitable amount of the thickening agent can be added. Indeed, in some embodiments, thickening agent (e.g., thrombin) is added to the multi-potent cells, plasma, and collagen a ratio of between about 1_{E}⁻¹⁰ and about 1_{E}¹⁰ (or any sub-range thereof) units of thrombin for every cc of the combination of multi-potent cells and plasma. In some other embodiments, thickening agent (e.g., thrombin) is added to the multi-potent cells, plasma, and collagen at any suitable sub-range of the aforementioned range (e.g., between about 1 and about 20,000 (or between about 4,000 and about 6,000) units of thickening agent for every cc of the combination of multi-potent cells and plasma in the wound packing). Indeed, in some embodiments, between about 100 and about 6,500 units of thrombin are added for every cc of the combination of multi-potent cells and plasma in the wound packing.

In some other embodiments, calcium chloride is added to the multi-potent cells, plasma, and collagen a ratio of between 1_{E}⁻¹⁰ and about 0.5 (or any sub-range thereof) moles of calcium chloride for every cc of the combination of multi-potent cells and plasma. In some other embodiments, calcium chloride is added to the multi-potent cells, plasma, and collagen at any suitable sub-range of the aforementioned range (e.g., between about 0.00001 and about 0.3 moles of thrombin for every cc of the combination of multi-potent cells and plasma in the wound packing). In one example, between about 4,000 and about 6,000 units of thrombin and between about 0.0001 moles and about 0.001 moles of calcium chloride are added to the wound packing for every cc of the combination of multi-potent cells and/or plasma in the packing.

The thickening agent can be added to the wound packing 150 at any suitable time, including without limitation, by being added to the wound packing after the multi-potent cells and/or plasma are added to the collagen, by being added to the collagen before the multi-potent cells and/or plasma are added to the collagen, and/or at any other suitable time. In some embodiments, however, the thickening agent is added after the multi-potent cells and plasma have been dispersed in the collagen.

In some embodiments, after the thickening agent (e.g., thrombin) has caused the wound packing to coagulate or otherwise harden, and/or after the wound packing (prior to and/or after application of an initial dose of thickening agent) is placed in the wound, an additional amount of thickening agent is added to the wound packing to help form a thicker barrier on one or more surfaces of the packing. In this regard, any suitable additional amount of one or more thickening agents can be added to the packing. Indeed, in some embodiments, once the wound packing has initially been treated with thickening agent, between about 1 and about 20,000 units (or any sub-range thereof) of the thickening agent are added to an outer surface of the wound packing. In one example, after the wound packing has been hardened with an initial application of the thickening agent, between about 20 and about 7,000 units of thrombin (or a sub-range thereof) are applied to an outer surface of the packing.

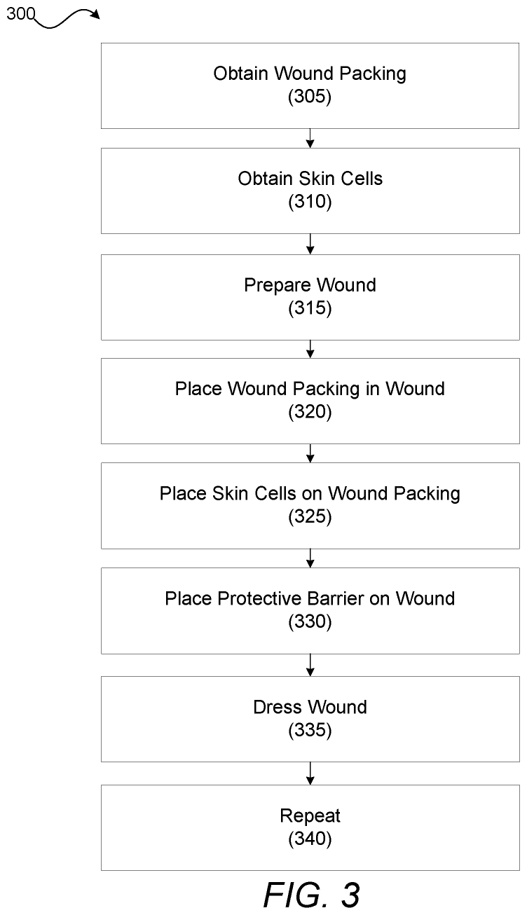

The described wound packing 150 can be used in any suitable manner. In one non-limiting example, FIG. 3 shows that, in some embodiments, the method 300 for using the wound packing includes obtaining the packing (as shown at step 305 and as discussed above). Step 310 of FIG. 3 further shows that, in some embodiments, the method 300 includes obtaining skin fragments for application to the wound packing and/or wound. In this regard, the skin fragments can be obtained from any suitable person, organism, and/or other source. Indeed, in some embodiments, the skin graft comprises an: autologous graft from the person who is to receive the wound packing, an isogenic graft from an identical twin of such recipient, an allogenic graft from another human, a xenogeneic graft from a non-human organism, and/or a synthetic material. In some embodiments, however, the skin fragments are taken as an autograft (which may include, without limitation, cultured epithelial autografts) from the patient that will be receiving the wound packing.

The skin fragments can also be obtained from any suitable portion of the donor's body. Some examples of suitable locations for obtaining such grafts include, but are not limited to, a person's back, one or more of a donor's legs, arms, hands, buttocks, and/or other suitable locations. Additionally, the skin grafts can be taken in any suitable manner, including, without limitation, through the use of a dermatome, expansion grafter, scalpel, razor blade, and/or another suitable instrument.

In some embodiments, the skin (e.g., epidermis and/or dermis) of the donor is grated, cut, chopped, micronized, and/or otherwise fragmented. In this regard, the skin fragments can be made to be any suitable size, including, without limitation, to have an average diameter and/or width that is less than about 2 cm (or any sub-range thereof). Indeed, in some embodiments, the skin fragments have an average diameter and/or width of between about 0.0000001 mm and about 2 cm (or any subrange thereof). In still other embodiments, the skin fragments have an average diameter and/or width of between 0.001 mm and about 5 mm.

Continuing with FIG. 3, step 315 shows that, in some embodiments, the described method 300 includes preparing a wound to receive the wound packing 150. In this regard, the wound can be prepared in any suitable manner that allows it to receive the wound packing, including, without limitation, by washing the wound, debriding the wound, disinfecting the wound, removing at least some necrotic tissue from the wound, causing the wound to bleed, applying an ointment to the wound, applying a disinfectant to the wound, and/or otherwise preparing the wound for healing. Although in some embodiments, the wound is debrided or otherwise prepared such that it has a bleeding base, in some other embodiments, the wound packing is applied without such a base.

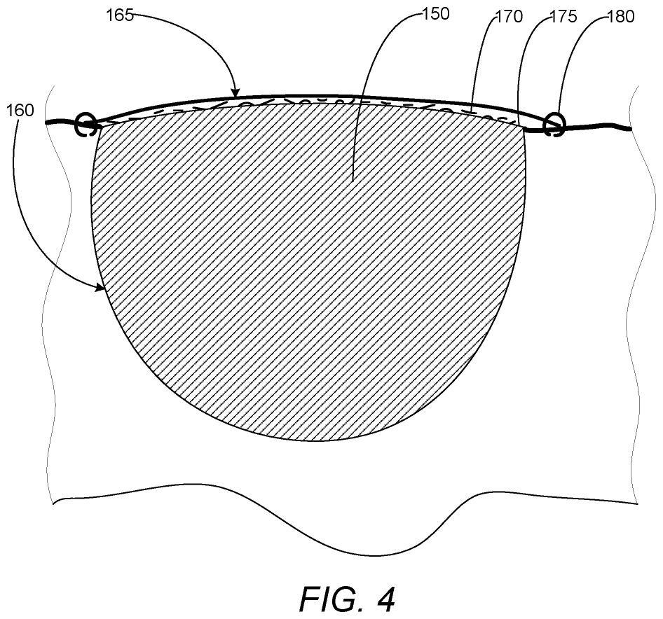

At step 320, FIG. 3 shows the wound packing 150 is placed in, placed on top of, and/or otherwise applied to the wound. While the wound packing can be applied to the wound in any suitable manner, in some embodiments, the packing (e.g., the collagen matrix) is sewn, stapled, glued, taped, bandaged in place, and/or otherwise secured to the wound. By way of non-limiting illustration, FIG. 4 shows a representative embodiment in which the wound packing 150 is placed in a wound 160 and then held in place by a protective barrier 165 (as described below).

Returning to FIG. 3, step 325 shows that some embodiments of the method 300 include the application of skin seeds or fragments to the wound packing 150. While the skin seeds can be applied to any portion of the wound 160 and/or wound packing (e.g., on an external surface of the packing, between a portion of the packing and an interior surface of the wound, inside the wound packing, just below a surface of the wound packing, and/or in any other suitable location), FIG. 4 shows that, in accordance with at least some embodiments, the skin fragments 170 are applied to an external surface 175 of the wound packing 150. In this example, skin fragments can, in some embodiments, help to epithelize the wound site, helping to close smaller wounds, helping larger wounds to approach a position in which they can be closed (e.g., via stitches, staples, bandages, another wound packing, etc.), and otherwise helping wounds to become more manageable.

While the amount of skin fragments that are to be placed on the wound packing 150 and/or wound 160 can vary from one wound to another, depending on the size, severity, and condition of the various wounds, in some embodiments, between about 0.1 mg and about 2 grams (or any sub-range thereof) of skin fragments are applied per cm² of the external surface area of the wound packing that is exposed from the wound. Indeed, in some embodiments, between about 0.1 grams and about 1 gram of skin fragments per cm² of external surface of the wound packing that is exposed from the wound.

Returning again to FIG. 3, step 330 shows that, in some embodiments, the method 300 optionally includes placing a protective barrier 165 over the wound packing 150 and wound 160. In this regard, some non-limiting examples of such barriers include a single layer protective membrane, such as a layer of a-cellular dermis (e.g., a-cellular dermis from a human, pig, monkey, and/or any other suitable organism), a layer of a surgical silicone, and/or any other suitable single layer membrane; a bilayer protective membrane, such as a piece of an a-cellular dermis with a basement membrane, a piece of an a-cellular dermis comprising a layer of surgical silicone, and/or any other suitable bi-layer membrane; and/or any other suitable membrane comprising two or more layers.

Where the wound packing 150 is covered with a protective barrier 165, the protective barrier can be attached to the wound 160 in any suitable manner, including, without limitation, via stitching, tape, staples, glue, a bandage, wrapping, and/or in any other suitable manner. By way of non-limiting illustration, FIG. 4 shows an embodiment in which the barrier 165 is attached to the wound 160 via a plurality of staples 180.

Returning again to FIG. 3, step 335 shows that, in some embodiments, the wound 160 and wound packing 150 is optionally dressed. In this regard, the wound can be dressed in any suitable manner, including, without limitation, by being dressed with one or more bandages, pieces of gauze, wrappings, total contact casts, non-weight-bearing casts and/or dressings, wound veils, contact layer flex bandages, compressive dressings, KERLIX™ wrappings, absorbent abdominal pads, calcium alginate applications, hydra fibers, fenestrated gauze drain sponges, TRIACT™ treatments (e.g., produced by Hollister of Libertyville, Ill., USA), ointments, petroleum-based jellies, and/or with any other suitable type of wound dressings.

Continuing with FIG. 3, step 340 shows that the wound 160 can be checked periodically, and that if the wound is healed in a desired period of time, one or more portions of the method 300 can be repeated. Indeed, in some embodiments, if a wound has not completely healed in a desired period of time (e.g., between about 3 days and about 2 months, or any sub-range thereof), another wound packing (e.g., a smaller wound packing where the wound has partially healed) can be added to the wound.

As previously mentioned, the described systems and methods can be modified in any suitable manner that allows the described wound packing 150 to be used to treat a wound 160. Indeed, in one example of a modification, in order to provide vascular stimulation that can further allow the wound to heal, multi-potent cells and/or plasma are injected into the individual around the wound.

In another example of a suitable modification, in some embodiments, the wound packing comprise multi-potent cells, plasma, and a thickening agent, but lacks collagen. In still another example of a suitable modification, some embodiments of the wound packing comprise plasma, collagen, and a thickening agent but lack multi-potent cells. In yet another example of a modification, some embodiments of the wound packing comprise multi-potent cells, collagen, and a thickening agent, but lack plasma.

Thus, the present invention relates to wound care. In particular, some implementations of the present invention relate to systems and methods for treating a wound. In particular, some implementations of the present invention relate to methods for forming a wound packing comprising multi-potent cells (e.g., bone marrow cells), plasma, collagen, and/or a thickening agent. In some further implementations, once the wound packing is applied to a wound, skin seeds are optionally applied to a surface of the packing and a protective barrier is applied over the packing.

In addition to any previously indicated modification, numerous other variations and alternative arrangements may be devised by those skilled in the art without departing from the spirit and scope of this description, and appended claims are intended to cover such modifications and arrangements. Thus, while the information has been described above with particularity and detail in connection with what is presently deemed to be the most practical and preferred aspects, it will be apparent to those of ordinary skill in the art that numerous modifications, including, but not limited to, form, function, manner of operation, and use may be made without departing from the principles and concepts set forth herein. Also, as used herein, the examples, implementations, and embodiments, in all respects, are meant to be illustrative only and should not be construed to be limiting in any manner. In addition, as the terms on, disposed on, attached to, connected to, coupled to, etc. are used herein, one object (e.g., a material, element, structure, member, etc.) can be on, disposed on, attached to, connected to, or coupled to another object—regardless of whether the one object is directly on, attached, connected, or coupled to the other object, or whether there are one or more intervening objects between the one object and the other object. Also, directions (e.g., on top of, below, above, top, bottom, side, up, down, under, over, upper, lower, lateral, medial, vertical, horizontal, distal, proximal, etc.), if provided, are relative and provided solely by way of example and for ease of illustration and discussion and not by way of limitation. Furthermore, where reference is made herein to a list of elements (e.g., elements a, b, c), such reference is intended to include any one of the listed elements by itself, any combination of less than all of the listed elements, and/or a combination of all of the listed elements. Also, as used herein, the terms a, an, and one may each be interchangeable with the terms at least one and one or more. It should also be noted, that while the term step is used herein, that term may be used to simply draw attention to different portions of the described methods and is not meant to delineate a starting point or a stopping point, or order for any portion of the methods, or to be limiting in any other way.

What is claimed is:

1. A wound packing comprising:
a mixture comprising a plasma selected from platelet rich plasma and plasma concentrate extracted from a patient and a material comprising multipotent cells extracted from the patient comprising bone marrow cells between about 1 and about 300 mls, wherein the multi-potent cells are combined with the plasma in the mixture at a ratio between about 100:1 and about 1:100 by volume; and
a thickening agent,
wherein the mixture and the thickening agent are mixed to form the wound packing, and
wherein the wound packing has at least one of a solid, a semi-solid, and a gelled consistency.
